(19) United States
(12) Patent Application Publication
(10) Pub. No.: US 2023/0277857 A1
(43) Pub. Date: Sep. 7, 2023

(54) SECURE REMOTE COMMUNICATION WITH A MEDICAL DEVICE

(71) Applicant: Boston Scientific Neuromodalation Corporation, Valencia, CA (US)

(72) Inventors: Keron Greene, Valencia, CA (US); Yue Li, Encino, CA (US); Chirag Shah, Valencia, CA (US); Joshua Uyeda, Los Angeles, CA (US)

(21) Appl. No.: 18/112,779

(22) Filed: Feb. 22, 2023

Related U.S. Application Data
(60) Provisional application No. 63/315,175, filed on Mar. 1, 2022.

(51) Int. Cl. A61N 1/372; A61N 1/362; H04W 12/037; G16H 40/67; H04L 9/08

(52) U.S. Cl. CPC A61N 1/37254; A61N 1/362; A61N 1/37235; A61N 1/37282; H04W 12/037; G16H 40/67; H04L 9/0819

(57) ABSTRACT

A system may include a user device, a clinician programmer and an implantable medical device. A first security protocol may be used to establish a first encrypted communication channel between the clinician programmer and the user device through cloud server(s). The first security protocol may authenticate entities and establish a first key set for encrypting and decrypting messages transmitted within the first encrypted communication channel. The user device and the implantable medical device may be configured to wirelessly communicate with each other through a secure wireless connection. A second security protocol may be used to establish a second encrypted communication channel, by establishing at least a first secret key, at least partially within the first encrypted communication channel. The second encrypted communication channel may extend between the implantable medical device and the clinician programmer. The first encrypted messages wrap the second encrypted messages.

### CLAIM OF PRIORITY

This application claims the benefit of U.S. Provisional Application No. 63/315,175, filed on Mar. 1, 2022, which is hereby incorporated by reference in its entirety.

### TECHNICAL FIELD

This document relates generally to medical systems, and more particularly, but not by way of limitation, to systems, devices, and methods that provide secure, remote communication with a medical device.

### BACKGROUND

Medical devices may include therapy-delivery devices configured to deliver a therapy to a patient and/or or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

After an initial activation of the device (e.g., implantation of an implantable device), the patient may be required to periodically visit the clinic in order to verify if their device is working correctly and programmed optimally. Device follow-ups may be performed by the clinicians and may be assisted by the sales representative from the device manufacturers. Device follow-ups are labor intensive and typically require patients to make multiple clinic visits. For example, SCS may have a very large number of programming options to temporally and spatially control the modulation field applied to the patient, and it may take significant time to evaluate the effectiveness of programmed therapy.

The present document discusses neuromodulation, also referred to as neurostimulation, as a specific example of a medical device. An implantable neuromodulation system may include an implantable neuromodulator attached to one or more implantable leads, where each lead may include one or more electrodes. The implantable neuromodulator delivers neuromodulation energy through one or more electrodes placed on or near a target site in the nervous system. An external programming device is commonly used to program the implantable neurostimulator with stimulation parameters controlling the delivery of the neurostimulation energy. Modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y). The values for these parameters may be customized to a patient. The modulation parameters may be configured as a neuromodulation program capable of being implemented by the neuromodulator, and the neuromodulator may be programmed with more than one program. In order to find a program that effectively provides a therapy (e.g., pain relief) with negligible side effects, the patient or clinician may implement different programs within the neuromodulator.

What is needed is a secure communication infrastructure that allows the medical device to communicate over long distances at virtually any time and location. Clinicians need a secure method for remotely communicating with medical devices, such as implantable medical devices, as they may need to modify therapy settings or otherwise interact with the medical device without middleman layers sniffing and/or modifying the data packets.

### SUMMARY

An example (e.g., “Example 1”) of a system configured to communicate via at least one cloud server, and may include a user device, a clinician programmer and an implantable medical device. The clinician programmer and the user device may be configured to establish, using a first security protocol, a first encrypted communication channel between the clinician programmer and the user device through the at least one cloud server. The first security protocol may authenticate entities and establish a first key set for encrypting and decrypting messages transmitted within the first encrypted communication channel. The user device and the implantable medical device may be configured to wirelessly communicate with each other through a secure wireless connection. The implantable medical device and the clinician programmer may be configured to establish, using a second security protocol, a second encrypted communication channel at least partially within the first encrypted communication channel. The second encrypted communicated channel may be established by establishing at least a first secret key for exchanging encrypted messages. The second encrypted communication channel may extend between the implantable medical device and the clinician programmer. The clinician programmer may be configured to program the implantable medical device by communicating with the user device using first encrypted messages within the first encrypted communication channel and communicating with the implantable device using encrypted messages within the second encrypted communication channel. The first encrypted messages wrap the second encrypted messages. The at least one cloud server does not know the first secret key and is unable to decipher messages between the clinician programmer and the implantable device using the first secret key.

In Example 2, the subject matter of Example 1 may optionally be configured such that the user device does not know the first secret key and is unable to decipher messages between the clinician programmer and the implantable device using the first secret key.

In Example 3, the subject matter of Example 1 may optionally be configured such that the first secret key may be used to exchange encrypted messages within the second encrypted communication channel between the clinician programmer and the user device, the second encrypted communication channel may be established by establishing a second secret key used to exchange encrypted messages between the implantable medical device and the user device, and the user device is configured to use the first secret key to decipher messages between the clinician programmer and the implantable medical device, and may be further configured to relay the deciphered messages to the implantable medical device using the second secret key.

In Example 4, the subject matter of any one or more of Examples 1-3 may optionally be configured such that the first security protocol includes Transport Layer Security (TLS).

In Example 5, the subject matter of any one or more of Examples 1-4 may optionally be configured such that the user device may include a smart phone or a tablet with a downloadable app configured to communicate over the first encrypted communication channel with the clinical programmer, configured to communicate over the secure wireless connection with the implantable medical device, and configured to relay messages between the implantable medical device and the clinical programmer using the second encrypted communication channel within the first encrypted communication channel.

In Example 6, the subject matter of any one or more of Examples 1-4 may optionally be configured such that the user device may include a remote control programming device configured to communicate over the first encrypted communication channel with the clinical programmer, configured to communicate over the secure wireless connection with the implantable medical device, and configured to relay messages between the implantable medical device and the clinical programmer using the second encrypted communication channel within the first encrypted communication channel.

In Example 7, the subject matter of any one or more of Examples 1-6 may optionally be configured such that the implantable medical device may be configured to deliver a therapy.

In Example 8, the subject matter of Example 7 may optionally be configured such that the implantable medical device may include a neuromodulator.

In Example 9, the subject matter of Example 8 may optionally be configured such that the neuromodulator may include a spinal cord stimulator (SCS), a deep brain stimulator (DBS), a peripheral nerve stimulator (PNS), a peripheral nerve field stimulator (PNFS), or a functional electric stimulator (FES).

In Example 10, the subject matter of Example 7 may optionally be configured such that the implantable medical device may include a cardiac stimulator configured to stimulate myocardia.

In Example 11, the subject matter of any one or more of Examples 7-10 may optionally be configured such that the implantable medical device may include at least one sensor configured to sense at least one health-related parameter.

In Example 12, the subject matter of any one or more of Examples 1-11 may optionally be configured such that the secure wireless connection may include inductive near-field communication, a Bluetooth connection, or a wireless local area network.

In Example 13, the subject matter of any one or more of Examples 1-12 may optionally be configured such that the first security protocol may use certificates to authenticate entities.

In Example 14, the subject matter of any one or more of Examples 1-13 may optionally be configured such that the second security protocol may authenticate the user device to communicate with the implantable medical device.

In Example 15, the subject matter of Example 14 may optionally be configured such that the authentication may include at least one of one or more passwords and two-factor or multi-factor authentication.

Example 16 includes subject matter (such as a method, means for performing acts, machine readable medium including instructions that when performed by a machine cause the machine to performs acts, or an apparatus to perform). The subject matter may be performed using a clinician programmer to remotely program an implantable medical device. The subject matter may include: establishing, using a first security protocol, a first encrypted communication channel between the clinician programmer and a user device through at least one cloud server, where the user device may be configured to wirelessly communicate with the implantable medical device through a secure wireless connection, and where the first security protocol may authenticate entities and may establish a first key set for encrypting and decrypting messages transmitted within the first encrypted communication channel; establishing, using a second security protocol, a second encrypted communication channel at least partially within the first encrypted communication channel, where the second encrypted communication channel may extend between implantable medical device and the clinician programmer, and where the establishing the second encrypted communicated channel may include establishing at least a first secret key for exchanging encrypted messages; and using the clinician programmer to program the implantable medical device by communicating between the clinician programmer and the user device using first encrypted messages within the first encrypted communication channel, and communicating between the clinician programmer and the implantable medical device using second encrypted messages within the second encrypted communication channel. The second encrypted messages may be wrapped within the first encrypted messages between the clinician programmer and the user device, where the at least one cloud server does not know the first secret key and is unable to decipher messages between the clinician programmer and the implantable device using the first secret key.

In Example 17, the subject matter of Example 16 may optionally be configured such that the user device does not know the first secret key and is unable to decipher messages between the clinician programmer and the implantable device using the first secret key.

In Example 18, the subject matter of Example 16 may optionally be configured such that the first secret key may be used to exchange encrypted messages within the second encrypted communication channel between the clinician programmer and the user device, the establishing the second encrypted communication channel may include establishing a second secret key used to exchange encrypted messages between the implantable medical device and the user device, and the user device may be configured to use the first secret key to decipher messages between the clinician programmer and the implantable medical device, and may be further configured to relay the deciphered messages to the implantable medical device using the second secret key.

In Example 19, the subject matter of any one or more of Examples 16-18 may optionally be configured such that the first security protocol may include Transport Layer Security (TLS).

In Example 20, the subject matter of any one or more of Examples 16-19 may optionally be configured such that the user device may include a smart phone or a tablet with a downloadable app in the smart phone or the tablet, where the using the clinician programmer to program the implantable medical device may include using the downloadable app to communicate over the first encrypted communication channel with the clinical programmer, communicate over the secure wireless connection with the implantable medical device, and relay messages between the implantable medical device and the clinical programmer using the second encrypted communication channel within the first encrypted communication channel.

In Example 21, the subject matter of any one or more of Examples 16-19 may optionally be configured such that the user device may include a remote control programming device configured to communicate over the first encrypted communication channel with the clinical programmer, where the using the clinician programmer to program the implantable medical device may include using the remote control programming device to communicate over the secure wireless connection with the implantable medical device, and relay messages between the implantable medical device and the clinical programmer using the second encrypted communication channel within the first encrypted communication channel.

In Example 22, the subject matter of any one or more of Examples 16-21 may optionally be configured such that the using the clinician programmer to program the implantable medical device may include programming the implantable medical device to deliver a therapy.

In Example 23, the subject matter of Example 22 may optionally be configured such that the implantable medical device may include a neuromodulator, and the therapy may include a spinal cord stimulation (SCS), a deep brain stimulation (DBS), a peripheral nerve stimulation (PNS), a peripheral nerve field stimulation (PNFS), or a functional electric stimulation (FES).

In Example 24, the subject matter of Example 22 may optionally be configured such that the implantable medical device may include a cardiac stimulator, and the therapy may include an electrical therapy to treat an arrhythmia or heart failure.

In Example 25, the subject matter of any one or more of Examples 16-24 may optionally be configured such that the implantable medical device may include at least one sensor configured to sense at least one health-related parameter.

In Example 26, the subject matter of any one or more of Examples 16-25 may optionally be configured such that the secure wireless connection may include inductive near-field communication, a Bluetooth connection, or a wireless local area network.

In Example 27, the subject matter of any one or more of Examples 16-26 may optionally be configured to further include using certificates to authenticate entities for the first encrypted communication channel.

In Example 28, the subject matter of any one or more of Examples 16-27 may optionally be configured to further include authenticating the user device to communicate with the implantable medical device for the second encrypted communication channel.

In Example 29, the subject matter of Example 28 may optionally be configured such that the authenticating the user device may include at least one of passwords and two-factor or multi-factor authentication.

Example 30 includes subject matter (such as a method, means for performing acts, machine readable medium including instructions that when performed by a machine cause the machine to performs acts, or an apparatus to perform). The subject matter may be performed using a clinician programmer to remotely program an implantable medical device. The subject matter may include: establishing, using a first security protocol including Transport Layer Security (TLS), a first encrypted communication channel between the clinician programmer and a user device through at least one cloud server, where the user device may include a smart phone or a tablet with a downloadable app in the smart phone or the tablet, where the user device may be configured to wirelessly communicate with the implantable medical device through a secure wireless connection, where the secure wireless connection may include inductive near-field communication, a Bluetooth connection, or a wireless local area network, and where the first security protocol may authenticate entities and establishes a first key set for encrypting and decrypting messages transmitted within the first encrypted communication channel; establishing, using a second security protocol, a second encrypted communication channel at least partially within the first encrypted communication channel, where the second encrypted communication channel may extend between implantable medical device and the clinician programmer, and where the establishing the second encrypted communication channel may include establishing at least a first secret key for exchanging encrypted messages; using the clinician programmer to program the implantable medical device by encrypting digital data using the second security protocol to create a second encrypted message, encrypting the second encrypted message using the first security protocol to create a first encrypted message, transmitting the first encrypted message to the user device, deciphering the first encrypted message at the user device to obtain the second encrypted message, and using the second encrypted message to program the implantable medical device.

In Example 31, the subject matter of Example 30 may optionally be configured such that the using the second encrypted message to program the implantable medical device may include transmitting the second encrypted message from the user device to the implantable medical device, and deciphering the second encrypted message at the implantable medical device to obtain the digital data used to program the implantable medical device, where the user device does not know the first secret key and is unable to decipher messages between the clinician programmer and the implantable device using the first secret key.

In Example 32, the subject matter of Example 30 may optionally be configured such that the using the second encrypted message to program the implantable medical device includes deciphering the second encrypted message using the first secret key at the user device to obtain the digital data, encrypting the digital data for wireless transmission to the medical device, wirelessly transmitting the encrypted digital data from the user device to the medical device using a wireless protocol, and deciphering the digital data using the second secret key at the medical device to obtain the digital data used to program the implantable medical device.

In Example 33, the subject matter of Example 32 may optionally be configured such that the first secret key may be used to exchange encrypted messages within the second encrypted communication channel between the clinician programmer and the user device, the establishing the second encrypted communication channel may include establishing a second secret key used to exchange encrypted messages between the implantable medical device and the user device, and the user device may be configured to use the first secret key to decipher messages between the clinician programmer and the implantable medical device, and may be further configured to relay the deciphered messages to the implantable medical device using the second secret key.

In Example 34, the subject matter of any one or more of Examples 30-33 may optionally be configured such that the implantable medical device may include a neuromodulator, and the therapy may include a spinal cord stimulation (SCS), a deep brain stimulation (DBS), a peripheral nerve stimulation (PNS), a peripheral nerve field stimulation (PNFS), or a functional electric stimulation (FES).

In Example 35, the subject matter of any one or more of Examples 30-33 may optionally be configured such that the implantable medical device may include a cardiac stimulator, and the therapy may include an electrical therapy to treat an arrhythmia or heart failure.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, a system for remotely communicating with a medical device.

FIG. 2 illustrates, by way of example and not limitation, a remote programming system for programing a therapy delivery device.

FIG. 3 illustrates, by way of example and not limitation, a healthcare monitoring system configured to remotely transfer data to a remote system.

FIG. 4 illustrates, by way of example and not limitation, a modulation device.

FIG. 5 illustrates, by way of example and not limitation, an embodiment of a remote programming system configured to provide secure channels to securely program an implanted medical device through a cloud.

FIG. 6 illustrates, by way of example and not limitation, the remote programming system of FIG. 5 in which exchanged messages between the clinician programmer and the implantable medical device are only understood by the clinician programmer and the implantable medical device.

FIG. 7 illustrates, by way of example and not limitation, an embodiment of the remote programming system of FIG. 5 in which a middleman application operating on the user device have access to a key to decrypt programming between the clinical programmer and the implantable medical device.

FIG. 8 illustrates, by way of example and not limitation, the remote programming system of FIG. 6 and an example process for aborting as programming session due to a lost connection.

FIG. 9 illustrates, by way of example and not limitation, the remote programming system of FIG. 6 and an example process for an unauthorized application in a user device.

FIG. 10 illustrates, by way of example and not limitation, a method for remotely programming a medical device such as using the remote programming system of FIG. 6.

FIG. 11 illustrates, by way of example and not limitation, a process for using the clinician programmer to program an implantable medical device such as using the remote programming system of FIG. 6.

FIG. 12 illustrates, by way of example and not limitation, a process for using the clinician programmer to program an implantable medical device which involves decrypting and re-encrypting at the user device, such as using the remote programming system of FIG. 7.

FIG. 13 illustrates, by way of example and not limitation, a method for remotely programming the medical device using the system of FIG. 6 and the process illustrated in FIG. 11 in which exchanged messages are only understood by the clinician programmer and the medical device.

FIG. 14 illustrates, by way of example and not limitation, a method for remotely programming the medical device using the system of FIG. 7 and the process illustrated in FIG. 12 in which exchanged messages are understood by the clinician programmer, the user device and the medical device.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter may employ a certificate-based, end-to-end encryption to provide secure communication that prevents devices from sniffing data packets during remote programming sessions or during other remote communication. Clinicians may use an application (e.g., clinician programmer) that communicates with a server (e.g., cloud server) used to relay information between the clinician and the medical device (e.g., stimulator) by way of another device (e.g., user device such as a phone or remote) serving as a “middleman.” More than one cloud server may be used to relay the information. Thus, as will be described in more detail below, the system for remote communication (or more specifically for remote programming by way of example) may include four main communication points, including a clinician application, cloud, a middleman application, and a medical device (e.g., implantable stimulator such as a cardiac stimulator or a neuromodulator). Certificate-based encryption/authentication mechanisms may be implemented to communicate between the user device and the clinician application through the cloud. For example, state-of-the art cryptographic protocols that provide encryption and authentication (e.g., Transport Layer Security (TLS)) may be used to establish secure communication between a clinician application and the cloud and between the cloud and a middleman application. TLS, which evolved from Secure Socket Layers (SSL), is commonly used in secure web browsing as it provides end-to-end security of data transmitted over the Internet between applications. TLS prevents information that is being transmitted from being seen by others. The middleman application may communicate with an implantable medical device over a short-range connection (e.g., a wireless connection such as Bluetooth Low Energy (BLE)) and relay encrypted messages between the clinician application and the implantable medical device. Certificate-based encryption/authentication mechanisms also may be implemented over the short-range connection between the user device and the medical device. According to some embodiments, no middleman layers (e.g., cloud, middleman application) can read or modify programming data exchanged between clinician programmer and the medical device. According to some embodiments, end-to-end encryption may be established between the clinical programmer and the middleman using one key and between middleman and the medical device using another key. A benefit may be significant cost and time savings, since patient reps will no longer have to travel and meet with patients for consultations, and patients will no longer have to travel to a clinic to have their stimulation parameters adjusted.

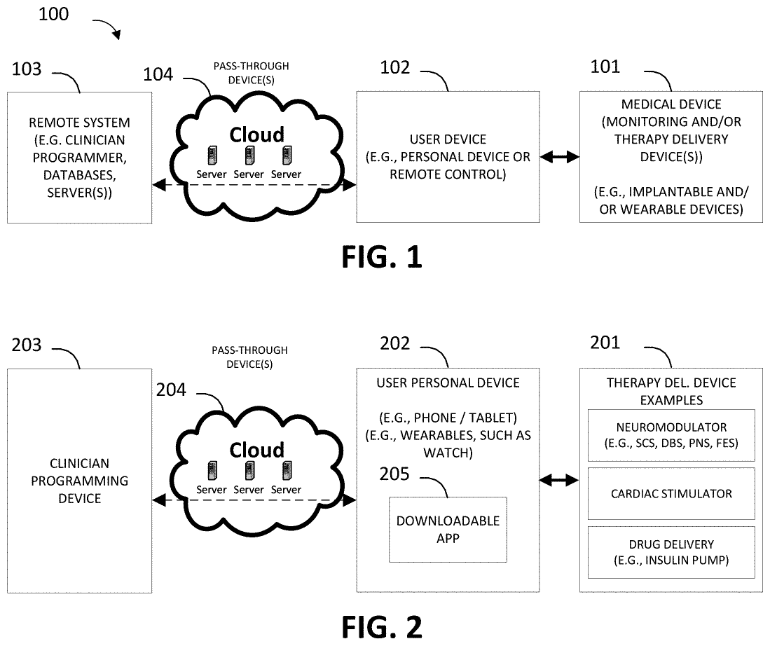

FIG. 1 illustrates, by way of example and not limitation, an embodiment of system for remotely communicating with a medical device. The system 100 may include a medical device 101, which may be an implantable device or may be a wearable device and may be configured to be a monitoring device and/or a therapy delivery device. Examples of monitoring devices include, but are not limited to, monitors that sense heart rate (or cardiac rhythm such as a Holter monitor)), blood pressure, posture, patient activity, or analytes (e.g., continuous glucose monitor). The monitor may be configured to receive various user input that may be relevant to the patient's condition, such as but not limited to diet, sleep, exercise, location and/or severity of pain, patient compliance to a therapy, and the like. The illustrated system 100 may include a user device 102, which may provide the user interface for monitor's user input. As such, the monitor functions may be performed using both the medical device 101 and the user device 102. Examples of therapy devices include electrical therapy devices such as neuromodulators and cardiac rhythm management devices, mechanical therapy devices, thermal therapy devices, and drug delivery devices. Examples of neuromodulators include, but are not limited to, spinal cord stimulators (SCS), deep brain stimulators (DBS), peripheral nerve stimulation (PNS) and function electrical stimulation (FES). Examples of cardiac rhythm management device include, but are not limited to, pacemakers and defibrillators. Examples of mechanical devices include, but are not limited to, devices configured to deliver compression to prevent deep vein thrombosis or to massage fluid from legs. Examples of drug delivery devices include, but are not limited to, insulin pumps or other infusion pumps. This disclosure discusses neuromodulation systems as a non-limiting example of a therapy device.

More particularly, FIG. 1 illustrates a system 100 that includes medical device 101, a user device 102 such as a phone, tablet or remote control configured to communicate with the medical device 101, and a remote system 103 such as a clinician programmer, database(s) such as may be used to upload monitored data from the medical device or user device, or server(s) used to download therapy programming or firmware/software updates to the medical device and/or user device. Communication between the user device 102 and the remote system 103 may pass through a number of device(s) or server(s) (e.g., “cloud” 104). The present subject matter may use certificate-based, end-to-end technology to ensure that the communication between the clinician programmer and the user device, or between the clinician programmer and the medical device, are not understood by any other device(s) through which the communication passes.

FIG. 2 illustrates, by way of example and not limitation, a remote programming system 200 for programing a therapy delivery device 201. The therapy-delivery device 201 may be, by way of example, a neuromodulator, a cardiac stimulator or a drug delivery device. By way of example and not limitation, the neuromodulation device may be configured to deliver SCS, DBS, PNS or FES. The therapy-delivery device may be a neuromodulation device configured to be electrically connected to electrodes and deliver neuromodulation energy, such as in the form of electrical pulses or other waveform, to the one or more neural targets though the electrodes. The system may also include sensing circuitry to sense a biological signal, which may but does not necessarily form a part of neuromodulation device. The delivery of the neuromodulation may be controlled using a plurality of modulation parameters that may specify the electrical waveform (e.g., pulses or pulse patterns or other waveform shapes) and a selection of the electrodes through which the electrical waveform is delivered. In various embodiments, at least some parameters of the plurality of modulation parameters are programmable by a user, such as a physician or other caregiver. For example, the parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude, pulse width, pulse rate, and burst rate. The remote system 203 may be configured to access and modify the user-programmable parameters, and may also provide the user with data indicative of the sensed biological signal or feature(s) of the sensed biological signal. The remote system 203 may include a user interface such as a graphical user interface (GUI) that allows the user to set and/or adjust values of the user-programmable modulation parameters. The user interface may also allow the user to view the data indicative of the sensed biological signal or feature(s) of the sensed biological signal and may allow the user to interact with that data. The neuromodulation device, the programming device and other devices or system may collect data that may be used by the neuromodulation system. For example, a user device 202 may have a user interface configured to enable the user to answer healthcare-related questions, such as but not limited to the efficacy of the therapy.

The therapy delivery device 201 may provide an open-loop therapy or a closed-loop therapy. Sensing circuitry may be configured for use to detect a biological signal for use to provide feedback for the closed-loop therapy. Sensing circuitry may include various components such as an application specific integrated circuit (ASIC), hardware and/or firmware. Sensing circuitry may include software implemented using a processor to further analyze feature(s) of the biological signal. The biological signal may be a measurable signal produced by electrical, chemical or mechanical activity. Examples of electrical signals may include sensing electrical activity in the brain (e.g., EEGs), electrical activity in nerves and muscles (e.g., EMGs), cardiac activity (e.g., ECGs), and other nerve sensing including both non-evoked responses and evoked responses (e.g., evoked compound action potentials (ECAPs) or evoked resonant nerve activity (ERNA)). Examples of mechanical signals may include sounds contractions detected via flex or strain sensors. Examples of chemical signals may include detected analyte concentrations such as glucose and the like. The system may include a feature detector that is configured to detect a plurality of available features of the biological signal. At least one of the features may be used as a closed-loop sensed feature of the biological signal, which may be used by a controller to provide a closed-loop therapy. The closed-loop sensed feature may be compared to a setpoint of that feature, and the difference may be fed into a feedback control algorithm.

The user device 202 may be a personal device of the user such as the user's smartphone, the user's tablet, or the user's wearable device such as a smart watch. The user may install a downloadable app 205 to be executed on the user device 202 to enable the user device to communicate with the therapy delivery device and to communicate with the remote, clinician programming device 203 through pass-through device(s) (“cloud” 204).

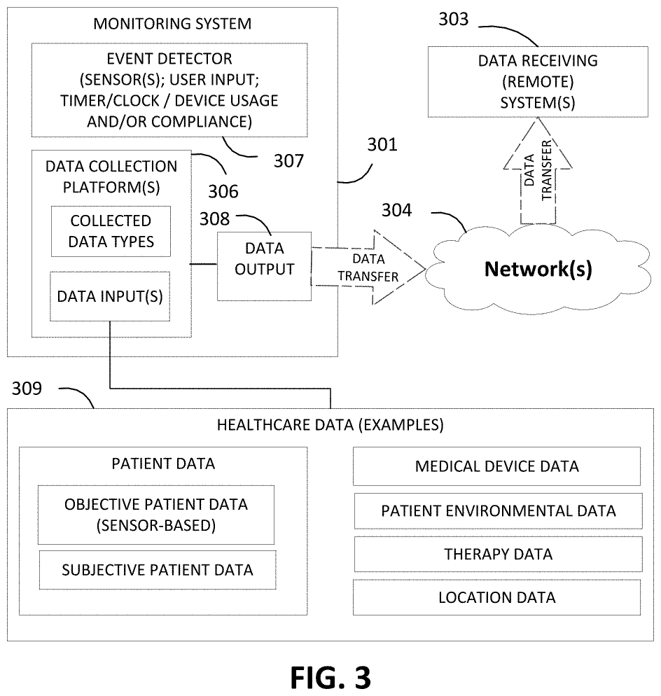

FIG. 3 illustrates, by way of example and not limitation, a healthcare monitoring system configured for use to collect healthcare-related data to be transferred to a remote system. The healthcare monitoring system 301 may be implanted, may be wearable, or may include both implanted and wearable components. The healthcare monitoring system may include the user device, using the user interface and other features of the user device (e.g., location data) to provide healthcare-related data. The monitoring system may be configured to transfer data to a remote data receiving system 303 for storage in a database for analysis, for example, through at least one network. The data transfer may use various network protocols to communicate and transfer data through one or more networks which may include the Internet (“cloud”) 304 and may include various wireless networks (e.g., Wi Fi) and/or short-range wireless technology such as Bluetooth which communication uses low-power radio waves between 2.400 GHz and 2.483.5 GHz. Bluetooth communication may implement security measures. For example, “pairing” equips each device with security keys, which can be used to encrypt data, and disguise an address/identity of the device. The pairing process may authenticate devices using codes. The data may be transferred directly from at least one of the external systems and/or may be transferred directly from at least one of the medical device(s). The external system(s) may be configured to receive data from the medical device(s) and/or receive data from other healthcare-related data source(s), and then transfer the data through the network(s) to the data receiving system(s).

The illustrated monitoring system 301 may include at least one data collection platform 306, an event detector 307, and a data output 308. The data collection platform(s) 306 may be configured to collect healthcare-related data and the data output 308 may be configured to transfer at least some of the collected data through the network(s) 304 to a data receiving system 303, which may include one or more server(s) or other systems remotely located from the patient. Thus, the data transfer may use various network protocols, including cryptographic protocols such as TLS, to communicate and transfer data through one or more networks which may include the Internet. The data collection platform(s) 306 may include at least one processor configured to execute instructions stored in memory (e.g., illustrated as processor(s)/memory) to perform processes to collect and transfer data. The event detector(s) 307 may be configured for detecting event(s), which may be used to determine when data is collected, or the data type/data resolution that is collected. The event detector 307 may detect event(s) using sensor(s), using user input(s), using a timer or clock, using indicator(s) of device usage, patient compliance with data collection and/or therapy, or various combinations thereof. Examples of healthcare data 309 may include patient data, medical device data, patient environmental data, therapy data, or various combinations thereof. The patient data may include objective data such as data collected from physiological sensor(s) and subjective data such as data collected from user-answered question(s) (e.g., “How do you rate your pain?”).

A monitoring system 301 may include medical device(s), external system(s) or other healthcare related data source(s) configured for use to collect healthcare-related data for transfer to a data receiving system. One or more of the medical device(s), external system(s) or other healthcare-related data source(s) may include technology used by the system to collect data, and thus may form part of the data collection platform. The data collection platform may be on one device or may be distributed among more than one device in the system. The monitoring system may include more than one medical device configured to communicate with each other or to an external system. Examples of medical devices include implantable and wearable devices. The medical device may be configured to only collect data, to only deliver therapy, or to both collect data and deliver therapy. The medical device may include sensor(s) configured for use to collect patient data (e.g., objective patient data). The medical device may be configured to collect and provide medical device data such as device model, configuration, settings, and the like. Thus, the medical device may be configured to collect patient data, medical device data, environmental data, and therapy data such as stimulation settings. Examples of external system(s) include remote controls, programmers, and personal devices such as phones, tablets, smart watches, personal computers, and the like. The external system(s) may include at least one user interface configured for use to receive user input, which may include user answers to questions. The user answers received via the user interface(s) may include subjective patient data (e.g., “How do you rate your pain?” or “Where do you feel pain?”) or objective patient data (e.g., “What is your heart rate?”, “What is your blood pressure?”, or “When did you fall asleep and wake up?”). The external system may be configured to collect medical device data from the medical device. Other healthcare-related data source(s) may include patient data received via a provider's server that stores patient health records. For example, patients may use a patient portal to access their health records such as test results, doctor notes, prescriptions, and the like. Other healthcare-related data sources may include various apps on a patient's phone or other device, or the data on a server accessed by those apps. By way of example and not limitation, this type of data may include heart rate, blood pressure, weight, respiration activity, muscle activity, analyte measurements (e.g., glucose measurements from a continuous glucose monitor), and the like. An app on a phone or patient's device may include or may be configured to access environmental data such as weather data and air quality information or location elevation data such as may be determined using cellular networks and/or a global positioning system (GPS). Weather data may include, but is not limited to, barometric pressure, temperature, sunny or cloud cover, wind speed, and the like.

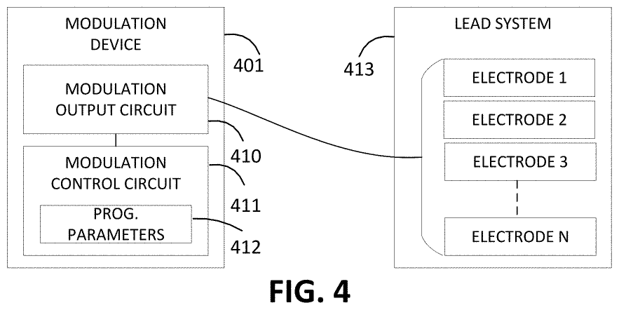

FIG. 4 illustrates an embodiment of a modulation device, which may be a specific example of the therapy delivery device of FIG. 2. The modulation device 401 may be an implantable device or an external device with leads percutaneously inserted to be positioned to stimulate a peripheral nerve. The illustrated embodiment of the modulation device 401 includes a modulation output circuit 410 and a modulation control circuit 411. The modulation device may include sensor(s) for patient monitoring and/or feedback control of the therapy, telemetry circuitry and power. The modulation output circuit 410 may produce and deliver the neuromodulation waveform (e.g., neuromodulation pulses). The modulation control circuit 411 may control the delivery of the neuromodulation pulses using programmable parameters 412. By way of example and not limitation, programmable parameters may include an electrode configuration (representing an electrode polarity being positive, negative, or zero) amplitude, pulse width, and rate (or frequency) of the electrical pulses, a fractionalized current distribution to the electrodes (as percentage cathodic current, percentage anodic current, or off), waveform shapes, and waveform patterns. The lead system 413 may include one or more leads each configured to be electrically connected to modulation device and a plurality of electrodes distributed in an electrode arrangement using the one or more leads. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between modulation output circuit and tissue of the patient. The neuromodulation pulses are each delivered from the modulation output circuit through a set of electrodes selected from the electrodes. The number of leads and the number of electrodes on each lead may depend on, for example, the distribution of target(s) of the neuromodulation and the need for controlling the distribution of electric field at each target. The neuromodulation system may be configured to therapeutically modulate the neural tissue by delivering the neuromodulation according to the programmable parameters. The therapeutic modulation may be supra-perception modulation or sub-perception modulation.

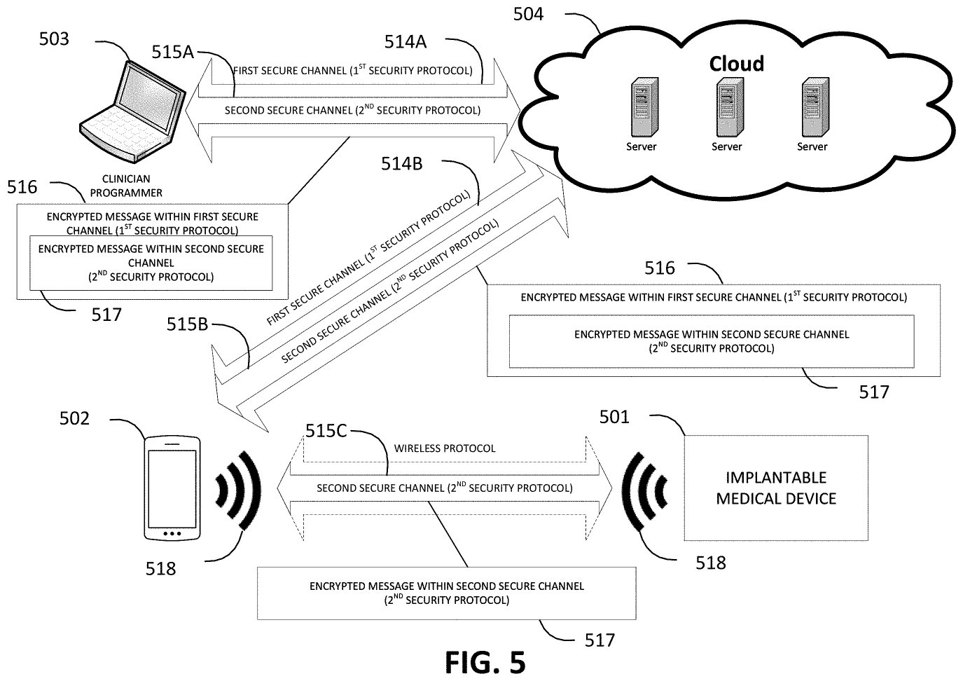

FIG. 5 illustrates, by way of example and not limitation, an embodiment of a remote programming system configured to provide secure channels to securely program an implanted medical device through a cloud. The illustrated remote programming system 500 includes a medical device 501 (e.g., implantable medical device), a user device 502 configured to communicate with the medical device, and a clinician programmer 503 configured to communicate with the user device via a cloud 504 of one or more servers that function as pass-through communication nodes between the clinician programmer and the user device. The present subject matter may provide secure communication, such as may include using the clinician programmer 503 to program the medical device 501. The secure communication may also involve communicating data from the medical device 501 and/or user device 502 back to the clinician programmer 503 or other remote system. This data from the medical device 501 and/or user device may be used to evaluate the patient's health and/or optimize the programmed therapy.

The present subject matter may use a certificate-based, end-to-end encryption to provide secure communication. Certificate-based authentication uses a digital certificate to identify a user, machine, or device before granting access to a resource, network, application, etc. to allow access only to approved users or devices, and prevent unauthorized users or devices. Both parties at the end of a communication link may be required to prove its identity before a connection can be made. Thus, digital certificates may be used to authenticate each device, including the intermediate device(s) in the cloud, used to transmit the communication. When a certificate-providing device provides its digital certificate, another device can verify the certificate with the certificate authority that issued it to confirm that the certificate-providing device is who it says it is. Encryption involves various processes to ensure the messages are unreadable in transit between network nodes for the purpose of providing data security over a shared communication channel. Encryption key(s) are established on the ends (e.g., “applications”) of intended communication channel so that only the applications operating on devices with the encryption key(s) are able to encrypt and decrypt the messages. A handshake process may be used to initiate secure communication between devices. For example, TLS (Transport Layer Security), which uses asymmetric encryption (public/private key), may perform the following in a handshake. A first device (e.g., “client”) may send to a second device (e.g., “server”) a “hello” message, including a string of random bytes and supported cipher suites, and the second device may reply by sending the second device's certificate, the second device's chosen cipher suite, and another random string of bytes. A cipher suite is a set of mathematical operations or algorithms used to establish the secure communication by making the communicated data appear random. The first device can verify the certificate with the certificate authority that issued it to confirm that the second device is who it says it is. The first device may use a public key, obtained from the second device, to encrypt random bytes, and the second device uses a private key to decrypt the random bytes. Both devices can generate session keys from the random bytes, and generated session keys can then be used to provide the encrypted communication.

The illustration presented in FIG. 5 shows that the system creates two secure communication channels. The illustrated first secure communication channel 514A and 514B, created using a first security protocol, extends between the clinician programmer 503 and the user device 502, and passes through server(s) within the cloud 504. Encryption key(s) may be established by the clinician programmer 503 and the user device 502 using the first security protocol, which may use a state-of-the-art technique such as TLS. The illustrated second secure communication channel 515A, 515B, 515C, created using a second security protocol, extends between the clinician programmer 503 and the medical device 501, and passes through server(s) within the cloud 504 and the user device 502. Encryption key(s) may be established by the clinician programmer 503 and the medical device 501 using the second security protocol. Both the first and second secure channels communicate encrypted messages, where messages encrypted in the first secure channel (first encrypted messages 516) are encrypted using the first security protocol, and messages encrypted in the second secure channel (second encrypted messages 517) are encrypted using the second security protocol. Notably, the second encrypted messages 517 are themselves encrypted as part of the first encrypted messages 516 for transmission through the first secure channel; i.e., the encryption in the first secure channel wraps the encryption in the second secure channel. Thus, intermediate devices used to transmit the communication using the first secure channel/first security protocol are unable to decrypt the messages encrypted using the second security protocol unless they have the key(s) associated with the second security protocol. A communication from the medical device 501 to the user device 502 over a wireless network 518 may use encrypted messages that are encrypted using the second security protocol 517. The encrypted message from the user device 502, through the cloud 504, and to the clinician programmer 503 may be encrypted using the first security protocol. Notably, the encrypted messages 516 using the first security protocol encrypts the encrypted messages 517 that were encrypted using the second security protocol.

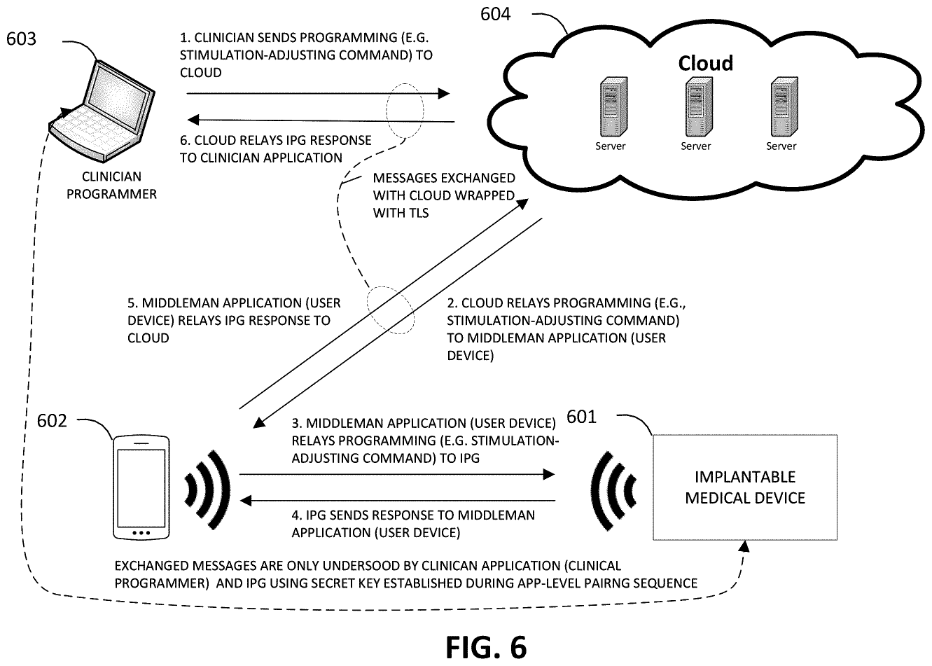

FIG. 6 illustrates, by way of example and not limitation, the remote programming system of FIG. 5 in which exchanged messages between the clinician programmer and the implantable medical device are only understood by the clinician programmer and the implantable medical device. That is, only the clinician programmer 603 and the medical device 601 have the encryption key(s) required to decrypt the encrypted messages transmitted between the medical device 601 to the clinician programmer 603. For example, a clinician may use the clinician programmer 603 to send programming (e.g., stimulation-adjusting command) to the cloud 604, the cloud 604 sends the programming to a user device 602 (or a middleman application operating on the user device), and the user device 602 relays the programming to the medical device 601 (e.g., implantable pulse generator or IPG) using a wireless communication protocol. The medical device 601 may send a response back to the user device 602 (middleman application), which relays the response to the cloud 604, which relays the response to the clinician programmer 603. TLS, or other state of the art technique for securing Internet communication, may be used to secure the communication between the user device 602 and the clinician programmer 603. The exchanged messages between the medical device 601 and the clinician programmer 603 are only understood by the medical device 601 and the clinician programmer 603 using one or more secret key(s) established during a pairing sequence. In the illustrated embodiment, the middleman application operating in the user device 602 does not have access to the secret key(s) and thus is unable to decrypt the messages. Similarly, the server(s) in the cloud 604 also do not have access to the secret key(s) established between the medical device 601 and the clinician programmer 603 and are unable to decrypt those messages.

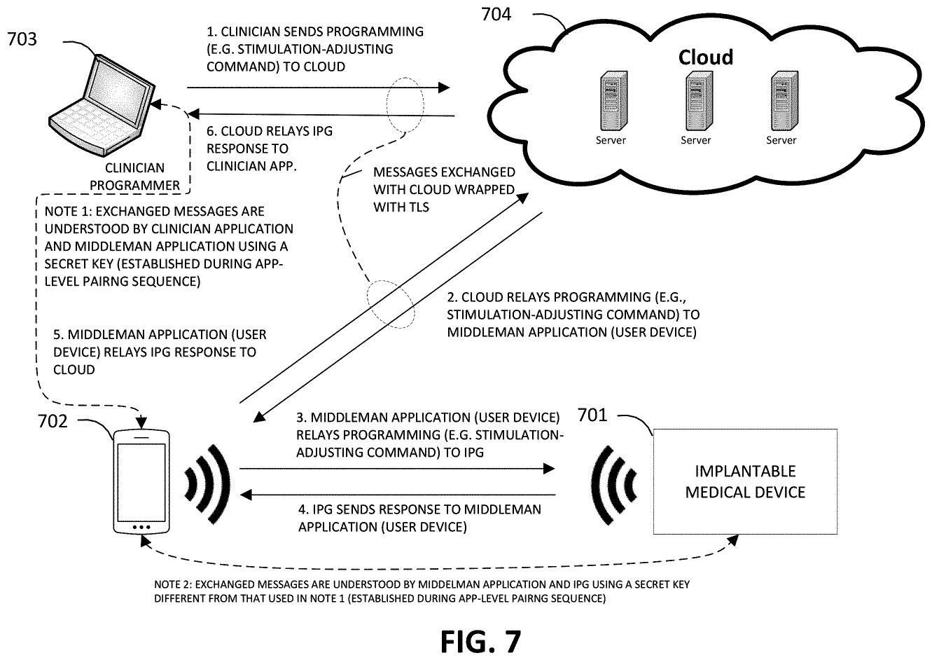

FIG. 7 illustrates, by way of example and not limitation, an embodiment of the remote programming system of FIG. 5 in which a middleman application operating on the user device 702 has access to a key to decrypt programming between the clinical programmer 703 and the implantable medical device 701. Thus, unlike the system illustrated in FIG. 6, the system illustrated in FIG. 7 is capable of understanding (e.g., decrypting) the messages exchanged between the medical device 701 and the clinician programmer 703. However, the messages continue to be wrapped with TLS or other security protocol, and thus the encrypted messages exchanged between the medical device 701 and the clinician programmer 703 are further encrypted in messages between the user device 702 and the clinician programmer 703. In some embodiments, the same key is used by both the medical device 701 and the user device 702 to decrypt communication. In some embodiments, different keys may be used by the medical device 701 and the user device 702 to decrypt communication.

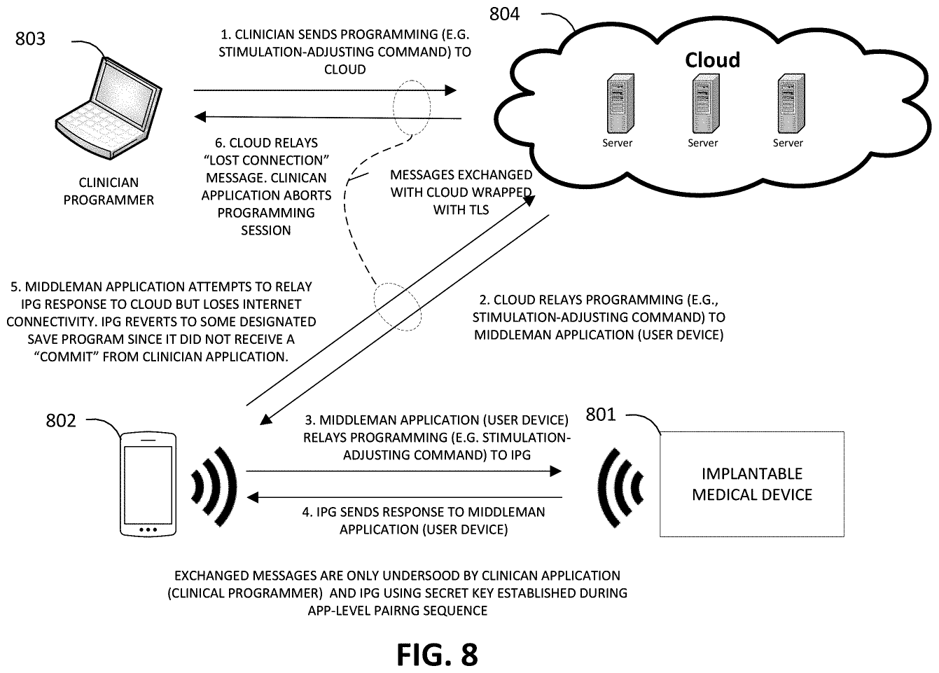

FIG. 8 illustrates, by way of example and not limitation, the remote programming system of FIG. 6 and an example process for aborting as programming session due to a lost connection. In the illustrated example, the process is similar to the process illustrated in FIG. 6. However, when the middleman application operating on the user device 802 attempts to relay the response from the medical device 801 to the cloud 804, the connection to the Internet may be lost. The cloud 804 may relay a “lost connection” to the clinician programmer 803, which may respond by aborting the programming session. The medical device 801 may revert to a designated program setting since the programming session was aborted before a “commit” was received from the clinician programmer 803.

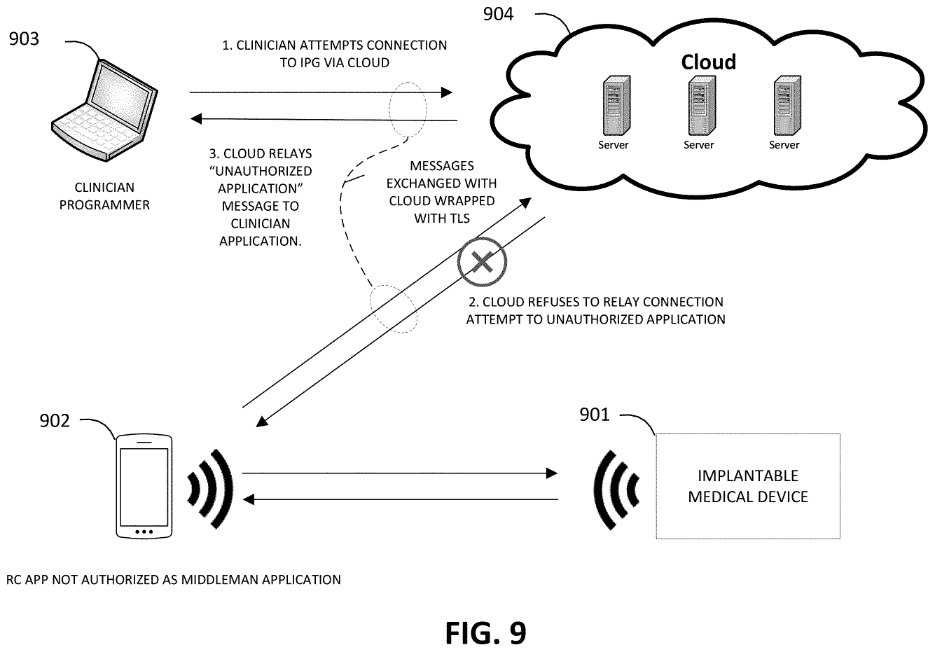

FIG. 9 illustrates, by way of example and not limitation, the remote programming system of FIG. 6 and an example process for an unauthorized application in a user device. In the illustrated system, the clinician programmer 903 may attempt to connect with the medical device 901 through the cloud 904, but the cloud 904 refuses to relay the connection to an application on the user device 902, which may be a remote control (RC), that has not been authorized. No programming communication will be delivered to the user device 902 for relaying the programming to the medical device 901. The cloud 904 may relay an “unauthorized application” message to the clinician application 903.

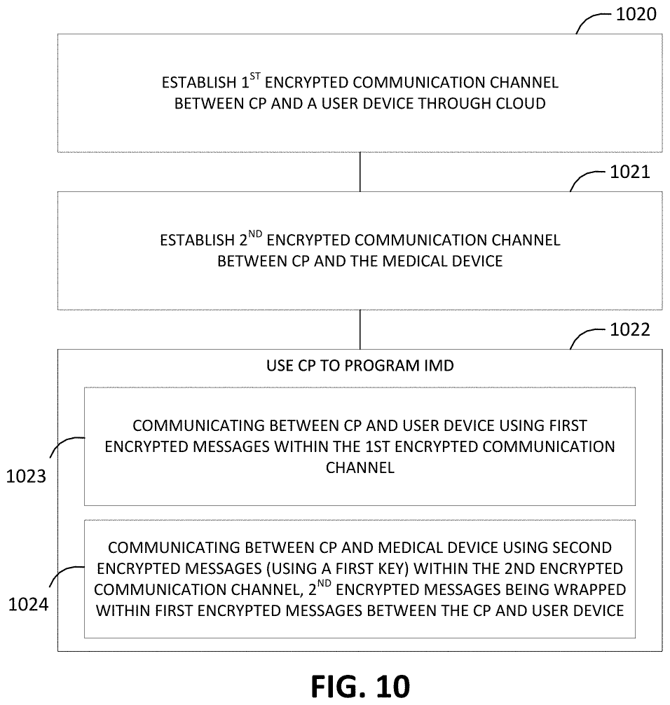

FIG. 10 illustrates, by way of example and not limitation, a method for remotely programming a medical device such as using the remote programming system of FIG. 6. The method may use a clinician programmer to remotely program an implantable medical device. The method may include, at 1020, establishing, using a first security protocol, a first encrypted communication channel between the clinician programmer and a user device through at least one cloud server. The user device may be configured to wirelessly communicate with the implantable medical device through a secure wireless connection. The first security protocol may authenticate entities and may establish a first key set for encrypting and decrypting messages transmitted within the first encrypted communication channel. The method may include, at 1021, establishing, using a second security protocol, a second encrypted communication channel at least partially within the first encrypted communication channel. The second encrypted communication channel may extend between implantable medical device and the clinician programmer. The second encrypted communicated channel may be established by establishing at least a first secret key for exchanging encrypted messages. At 1022, the clinician programmer may be used to program the implantable medical device by communicating between the clinician programmer and the user device using first encrypted messages within the first encrypted communication channel 1023, and communicating between the clinician programmer and the implantable medical device using second encrypted messages within the second encrypted communication channel 1024. The second encrypted messages are wrapped within the first encrypted messages between the clinician programmer and the user device. The cloud server(s) does not know the first secret key and is unable to decipher messages between the clinician programmer and the implantable device using the first secret key.

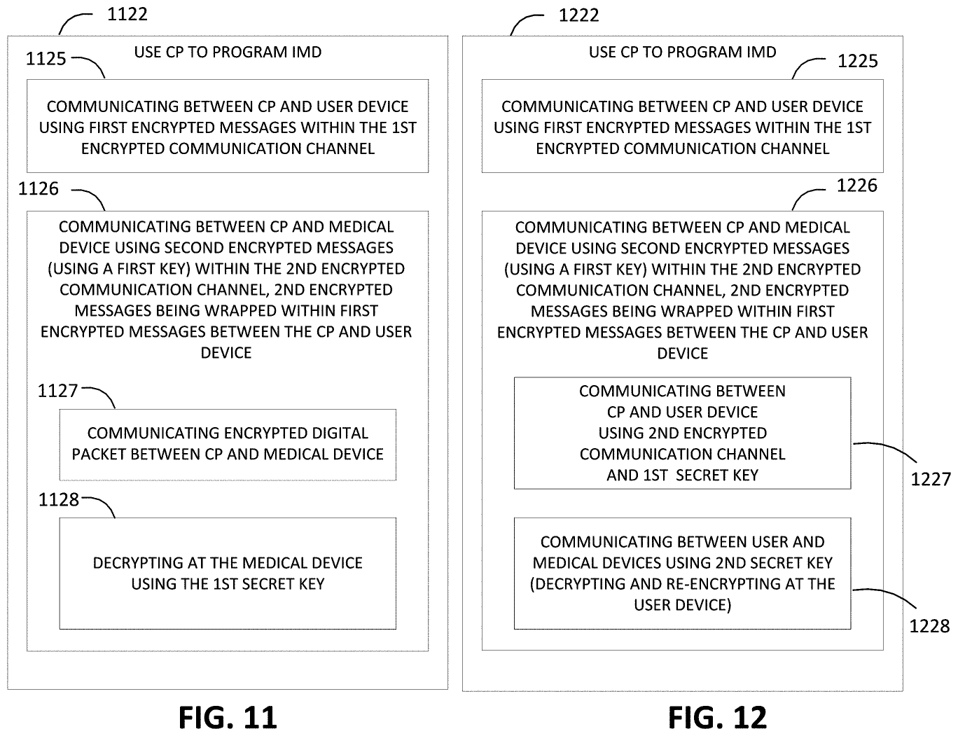

FIG. 11 illustrates, by way of example and not limitation, a process for using the clinician programmer to program an implantable medical device such as using the remote programming system of FIG. 6. The process 1122, generally corresponding to process 1022 in FIG. 10, may include communicating between the clinician programmer and the user device using first encrypted messages within the first encrypted communication channel 1125, and communicating between the clinician programmer and the medical device using second encrypted messages within the second encrypted communication channel 1126. The second encrypted messages are encrypted using a first secret key, and are wrapped within the first encrypted messages between the clinician programmer and the user device. The communicating with the second encrypted messages may include communicating encrypted digital packets between the clinician programmer and the medical device 1127, and decrypting the encrypted digital packet at the medical device using the first secret key 1128.

FIG. 12 illustrates, by way of example and not limitation, a process for using the clinician programmer to program an implantable medical device which involves decrypting and re-encrypting at the user device, such as using the remote programming system of FIG. 7. The process 1222, generally corresponding to process 1022 in FIG. 10, may include communicating between the clinician programmer and the user device using first encrypted messages within the first encrypted communication channel 1225, and communicating between the clinician programmer and the medical device using second encrypted messages within the second encrypted communication channel 1226. The second encrypted messages are encrypted using a first secret key, and are wrapped within the first encrypted messages between the clinician programmer and the user device. The communicating with the second encrypted messages 1226 may include communicating between the clinician programmer and the user device using the second encrypted communication channel and a first secret key 1227, and may include communicating between the user device and the medical device using the second secret key 1228. The user device, having knowledge of the first secret key, may be configured to decrypt and re-encrypt the communications. In some embodiments, one key may serve as both the first and second secret keys; and in some embodiments, different keys may serve as the first and second secret keys.

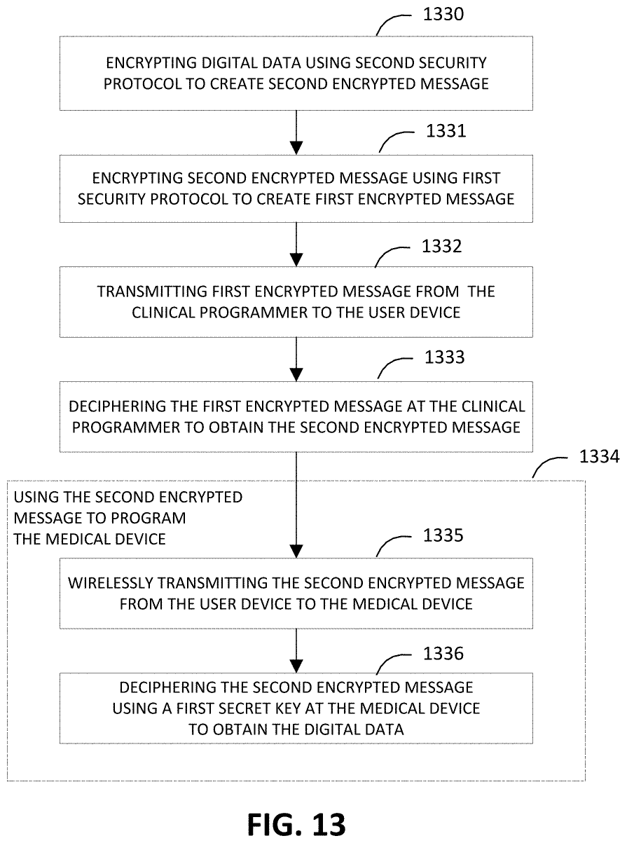

FIG. 13 illustrates, by way of example and not limitation, a method for remotely programming the medical device using the system of FIG. 6 and the process illustrated in FIG. 11 in which exchanged messages are only understood by the clinician programmer and the medical device. The process may include encrypting digital data using the second security protocol to create a second encrypted message 1330, encrypting the second encrypted message using the first security protocol to create a first encrypted message 1331, transmitting the first encrypted message to the user device 1332, deciphering the first encrypted message at the user device to obtain the second encrypted message 1333, and using the second encrypted message to program the implantable medical device 1334. The second encrypted message may be used to program the medical device 1334 by wirelessly transmitting the second encrypted message from the user device to the medical device 1335, and deciphering the second encrypted message using a first secret key at the medical device to obtain the digital data 1336.

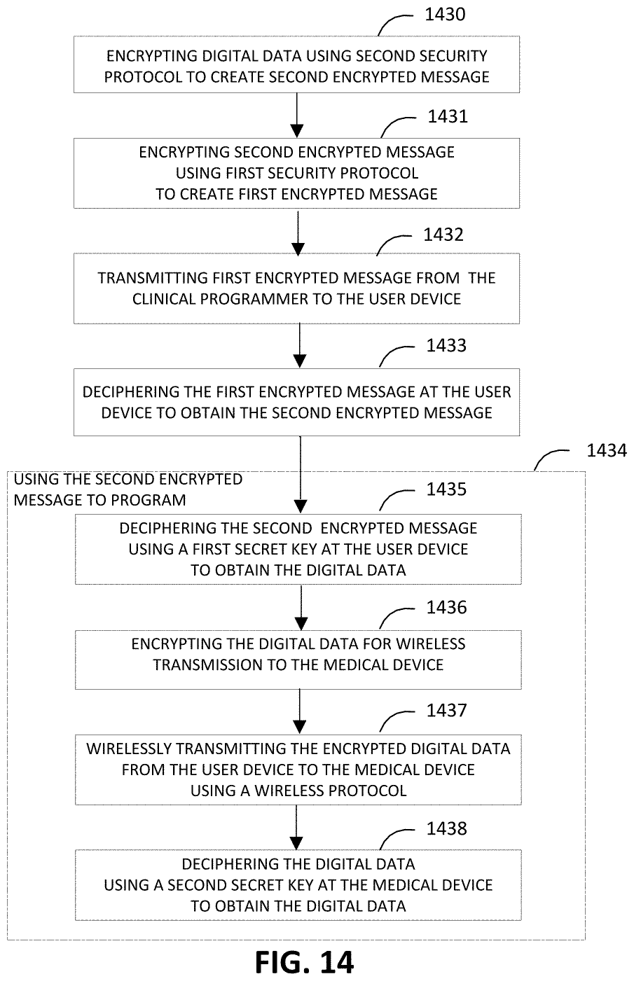

FIG. 14 illustrates, by way of example and not limitation, a method for remotely programming the medical device using the system of FIG. 7 and the process illustrated in FIG. 12 in which exchanged messages are understood by the clinician programmer, the user device and the medical device. The process may include encrypting digital data using the second security protocol to create a second encrypted message 1430, encrypting the second encrypted message using the first security protocol to create a first encrypted message 1431, transmitting the first encrypted message to the user device 1432, deciphering the first encrypted message at the user device to obtain the second encrypted message 1433, and using the second encrypted message to program the implantable medical device 1434. The second encrypted message may be used to program the medical device 1434 by deciphering the second encrypted message using the first secret key at the user device to obtain the digital data 1435, encrypting the digital data for wireless transmission to the medical device 1436, wirelessly transmitting the encrypted digital data from the user device to the medical device using a wireless protocol 1437, and deciphering the digital data using the second secret key at the medical device to obtain the digital data used to program the implantable medical device 1438.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system configured to communicate via at least one cloud server, comprising:
a user device,
a clinician programmer, wherein the clinician programmer and the user device are configured to establish, using a first security protocol, a first encrypted communication channel between the clinician programmer and the user device through the at least one cloud server, wherein the first security protocol authenticates entities and establishes a first key set for encrypting and decrypting messages transmitted within the first encrypted communication channel; and
an implantable medical device, wherein the user device and the implantable medical device are configured to wirelessly communicate with each other through a secure wireless connection, wherein the implantable medical device and the clinician programmer are configured to establish, using a second security protocol, a second encrypted communication channel at least partially within the first encrypted communication channel, wherein the second encrypted communicated channel is established by establishing at least a first secret key for exchanging encrypted messages, and wherein the second encrypted communication channel extends between implantable medical device and the clinician programmer,
wherein the clinician programmer is configured to program the implantable medical device by communicating with the user device using first encrypted messages within the first encrypted communication channel and communicating with the implantable device using encrypted messages within the second encrypted communication channel, the first encrypted messages wrapping the second encrypted messages, wherein the at least one cloud server does not know the first secret key and is unable to decipher messages between the clinician programmer and the implantable device using the first secret key.

2. The system of claim 1, wherein the user device does not know the first secret key and is unable to decipher messages between the clinician programmer and the implantable device using the first secret key.

3. The system of claim 1, wherein:
the first secret key is used to exchange encrypted messages within the second encrypted communication channel between the clinician programmer and the user device,
the second encrypted communication channel is established by establishing a second secret key used to exchange encrypted messages between the implantable medical device and the user device, and
the user device is configured to use the first secret key to decipher messages between the clinician programmer and the implantable medical device, and is further configured to relay the deciphered messages to the implantable medical device using the second secret key.

4. The system of claim 1, wherein the first security protocol includes Transport Layer Security (TLS).

5. The system of claim 1, wherein the user device includes a smart phone or a tablet with a downloadable app configured to communicate over the first encrypted communication channel with the clinical programmer, configured to communicate over the secure wireless connection with the implantable medical device, and configured to relay messages between the implantable medical device and the clinical programmer using the second encrypted communication channel within the first encrypted communication channel.

6. The system of claim 1, wherein the user device includes a remote control programming device configured to communicate over the first encrypted communication channel with the clinical programmer, configured to communicate over the secure wireless connection with the implantable medical device, and configured to relay messages between the implantable medical device and the clinical programmer using the second encrypted communication channel within the first encrypted communication channel.

7. The system of claim 1, wherein the implantable medical device is configured to deliver a therapy.

8. The system of claim 7, wherein the implantable medical device includes a neuromodulator.

9. The system of claim 8, wherein the neuromodulator includes a spinal cord stimulator (SCS), a deep brain stimulator (DBS), a peripheral nerve stimulator (PNS), a peripheral nerve field stimulator (PNFS), or a functional electric stimulator (FES).

10. The system of claim 7, wherein the implantable medical device includes a cardiac stimulator configured to stimulate myocardia.

11. The system of claim 7, wherein the implantable medical device includes at least one sensor configured to sense at least one health-related parameter.

12. The system of claim 1, wherein the secure wireless connection includes inductive near-field communication, a Bluetooth connection, or a wireless local area network.

13. The system of claim 1, wherein the first security protocol uses certificates to authenticate entities.

14. The system of claim 1, wherein the second security protocol authenticates the user device to communicate with the implantable medical device.

15. The system of claim 14, wherein the authentication includes at least one of one or more passwords and two-factor or multi-factor authentication.

16. A method for using a clinician programmer to remotely program an implantable medical device, the method comprising:
establishing, using a first security protocol, a first encrypted communication channel between the clinician programmer and a user device through at least one cloud server, wherein the user device is configured to wirelessly communicate with the implantable medical device through a secure wireless connection, wherein the first security protocol authenticates entities and establishes a first key set for encrypting and decrypting messages transmitted within the first encrypted communication channel;
establishing, using a second security protocol, a second encrypted communication channel at least partially within the first encrypted communication channel, wherein the second encrypted communication channel extends between implantable medical device and the clinician programmer, wherein the establishing the second encrypted communicated channel includes establishing at least a first secret key for exchanging encrypted messages; and
using the clinician programmer to program the implantable medical device by communicating between the clinician programmer and the user device using first encrypted messages within the first encrypted communication channel, and communicating between the clinician programmer and the implantable medical device using second encrypted messages within the second encrypted communication channel, the second encrypted messages being wrapped within the first encrypted messages between the clinician programmer and the user device, wherein the at least one cloud server does not know the first secret key and is unable to decipher messages between the clinician programmer and the implantable device using the first secret key.

17. The method of claim 16, wherein the user device does not know the first secret key and is unable to decipher messages between the clinician programmer and the implantable device using the first secret key.

18. The method of claim 16, wherein:
the first secret key is used to exchange encrypted messages within the second encrypted communication channel between the clinician programmer and the user device,
the establishing the second encrypted communication channel includes establishing a second secret key used to exchange encrypted messages between the implantable medical device and the user device, and
the user device is configured to use the first secret key to decipher messages between the clinician programmer and the implantable medical device, and is further configured to relay the deciphered messages to the implantable medical device using the second secret key.

19. The method of claim 16, wherein the using the clinician programmer to program the implantable medical device includes programming the implantable medical device to deliver a therapy.

20. A method for using a clinician programmer to remotely program an implantable medical device, the method comprising:
establishing, using a first security protocol including Transport Layer Security (TLS), a first encrypted communication channel between the clinician programmer and a user device through at least one cloud server, wherein the user device includes a smart phone or a tablet with a downloadable app in the smart phone or the tablet, wherein the user device is configured to wirelessly communicate with the implantable medical device through a secure wireless connection, wherein the secure wireless connection includes inductive near-field communication, a Bluetooth connection, or a wireless local area network, and wherein the first security protocol authenticates entities and establishes a first key set for encrypting and decrypting messages transmitted within the first encrypted communication channel;
establishing, using a second security protocol, a second encrypted communication channel at least partially within the first encrypted communication channel, wherein the second encrypted communication channel extends between implantable medical device and the clinician programmer, wherein the establishing the second encrypted communication channel includes establishing at least a first secret key for exchanging encrypted messages; and
using the clinician programmer to program the implantable medical device by:
encrypting digital data using the second security protocol to create a second encrypted message;
encrypting the second encrypted message using the first security protocol to create a first encrypted message;
transmitting the first encrypted message to the user device;
deciphering the first encrypted message at the user device to obtain the second encrypted message; and
using the second encrypted message to program the implantable medical device.
